(19) United States
(12) Patent Application Publication
(10) Pub. No.: US 2004/0093027 A1
(43) Pub. Date: May 13, 2004

(54) Barbed tissue connector for sealing vascular puncture wounds

(76) Inventors: Walter Fabisiak, Highland Mills, NY (US); Lillian Quintero, Chester, NY (US)

Correspondence Address: J. Gary Mohr, Datascope Corp, 14 Philips Parkway, Montvale, NJ, 07645, US

(21) Appl. No.: 10/379,349

(22) Filed: Mar. 4, 2003

Related U.S. Application Data
(60) Provisional application No. 60/361,479, filed on Mar. 4, 2002.

(51) Int. Cl. A61B 17/08

(52) U.S. Cl. 606/215

(57) ABSTRACT

A method and device for sealing a blood vessel puncture by means of a self-anchoring, barbed tissue connector.

### RELATED APPLICATIONS

This application herein incorporates by reference and claims priority to provisional application No. 60/361,479 filed Mar. 4, 2002.

### BACKGROUND OF THE INVENTION

1. Field of the Invention

The invention relates to a method and device for sealing a vascular puncture wound. More particularly, the invention relates to a method and device for sealing a vascular puncture wound incorporating a barbed tissue connector.

2. Description of the Prior Art

In certain medical procedures, such as cardiac catheterization, dilation and counterpulsation, a catheter or other device is inserted into an artery, most commonly by percutaneous methods, and then fed through the arterial tree to the site where needed, frequently, the region of the heart. The site usually selected for insertion of the catheter is the groin, because the femoral artery in that region is relatively easy to locate.

These procedures are normally initiated by insertion of an angiographic needle, followed by passing a guide wire through that needle into the artery. The needle is then removed leaving the guide wire in place. Next, a sheath-dilator set is passed over the guide wire into the artery in order to enlarge the opening sufficiently to permit entry of the catheter or other device. The dilator is then removed, leaving the sheath or guide cannula in place. The catheter or other device can then be inserted through the cannula with full confidence that when it emerges from the distal end it will be within the lumen of the artery.

It should be understood that the subject invention is independent of the blood vessel involved. While it is expected that the femoral artery will be the most commonly used blood vessel, use of other arteries as well as veins is anticipated as well.

After a procedure, such as counterpulsation, has been completed, the sheath must be removed and the wound closed. Often, this can be accomplished simply by the application of digital pressure, generally augmented by the use of a pressure dressing. Customarily, pressure must be applied for at least ½ hour, and frequently for much longer than that. While pressure dressings often suffice, it is not uncommon for additional devices, such as sandbags, to be needed. In addition, during this period the patient must be immobilized, lest movement interfere with the closing process. Because of the pressure required, the time during which it must be applied and the need for immobilization, the procedure is painful and uncomfortable. The procedure also requires prolonged personal attention of a health care professional. Finally, wound closures accomplished in this manner are prone to reopen unexpectedly long after closure appears to have been completed. Patients are therefore often required to remain in the hospital for 24 hours or longer.

A device for sealing the wound, overcoming many of the above mentioned problems, disclosed in U.S. Pat. Nos. 5,871,501, 5,853,421, 5,830,130, 5,741,223, 5,728,122, 5,725,498, 5,591,204, 5,437,631, 5,391,183, and 5,310,407, all herein incorporated by reference in their entirety, is known in the medical field under Datascope Corp.'s VasoSeal® trademark. The VasoSeal® product comprises a charge of hemostatic material and a hollow sheath adapted to pass through the tissue channel of a patient, the sheath having a cross sectional profile larger than the puncture in the patient's blood vessel. The VasoSeal® product uses a locating guide wire to locate the blood vessel and then places the hemostatic material in the hollow sheath and advances the hemostatic material through the sheath to the outside of the vessel wall around the puncture. The hemostatic material is a compressed cylinder or plug of collagen, which swells slightly when the plug contacts the exterior wall of the blood vessel.

U.S. Pat. No. 5,871,501, herein incorporated by reference, discloses an improvement on the VasoSeal® product. The improvement comprises the use of a locating guide wire that prevents entrance of the collagen plug into the blood vessel so long as a sufficient amount of tension is applied to the guide wire during advancement of the plug into the tissue tract. In order to prevent bleeding from the puncture during the procedure, the blood vessel is pinched through the manual application of pressure external to the patient over the blood vessel.

The prior art arterial closure method described above has proven very successful in reducing the arterial sealing period, and thus, in enhancing the comfort and mobility of thousands of patients. However, there is always the need to minimize the possibility of plug entrance into the blood vessel.

Entrance of the plug into the artery is dangerous for a number of reasons. Entrance of the distal tip of the plug, or the entire plug, into the artery obstructs blood passage in the artery and emboli formation on the plug further obstructs blood flow. Furthermore, emboli may break off the distal tip of the plug and flow downstream. Free floating emboli must be removed surgically to prevent decreased circulation to distal extremities.

While the present arterial closure devices may be suitable for the particular purpose employed, or for general use, they are not as suitable for the purposes of the present invention as disclosed hereafter.

### SUMMARY OF THE INVENTION

The invention is a method and device for sealing a blood vessel puncture by means of a self-anchoring, barbed tissue connector.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is side view of the blood vessel sealing device of the present invention inserted into a tissue tract of a patient, shown in cross section.

FIG. 2 is a side view of the blood vessel sealing device of FIG. 1 with the hollow tubes deployed.

FIG. 3 is a side view of the blood vessel sealing device of FIG. 1 with the hollow tubes and snare means deployed.

FIG. 4 is a side view of the blood vessel sealing device of FIG. 1 with the hollow tubes and snare means deployed and with the barbed tissue connector deployed and partially passing through a snare at a distal end of the snare means.

FIG. 5 is a side view of the blood vessel sealing device of FIG. 4 with the snare means partially retracted and tightened over barbed tissue connector.

FIG. 6 is a side view of the blood vessel sealing device of FIG. 4 with the snare means and distal end of the barbed tissue connector retracted into the hollow tube from which the snare means was deployed.

FIG. 7 is a side view of the blood vessel sealing device with the hollow tubes retracted and with the barbed tissue connector spanning the puncture.

FIG. 8 is a side view of the barbed tissue connector spanning the sealed blood vessel puncture and extending out of the tissue tract.

FIG. 9A is a side view of a first embodiment of a barbed tissue connector.

FIG. 9B is a side view of a second embodiment of a barbed tissue connector.

FIG. 9C is a side view of a third embodiment of a barbed tissue connector.

FIG. 10 is a side view of barbed tissue connector spanning the sealed blood vessel puncture with a collagen plug disposed over the barbed tissue connector in the tissue tract.

### DETAILED DESCRIPTION OF THE PREFERRED EMBODIMENTS

In certain medical procedures, such as cardiac catheterization, dilation and counterpulsation, a catheter or other device is inserted into an artery, most commonly by percutaneous methods, and then fed through the arterial tree to the site where needed, frequently, the region of the heart. The site usually selected for insertion of the catheter is the groin, because the femoral artery in that region is relatively easy to locate.

These procedures are normally initiated by insertion of an angiographic needle, followed by passing a guide wire through that needle into the artery. The needle is then removed leaving the guide wire in place. Next, a sheath-dilator set is passed over the guide wire into the artery in order to enlarge the opening sufficiently to permit entry of the catheter or other device. The dilator is then removed, leaving the sheath or guide cannula in place. The catheter or other device can then be inserted through the cannula with full confidence that when it emerges from the distal end it will be within the lumen of the artery.

It should be understood that the subject invention is independent of the blood vessel involved. While it is expected that the femoral artery will be the most commonly used blood vessel, use of other arteries as well as veins is anticipated as well.

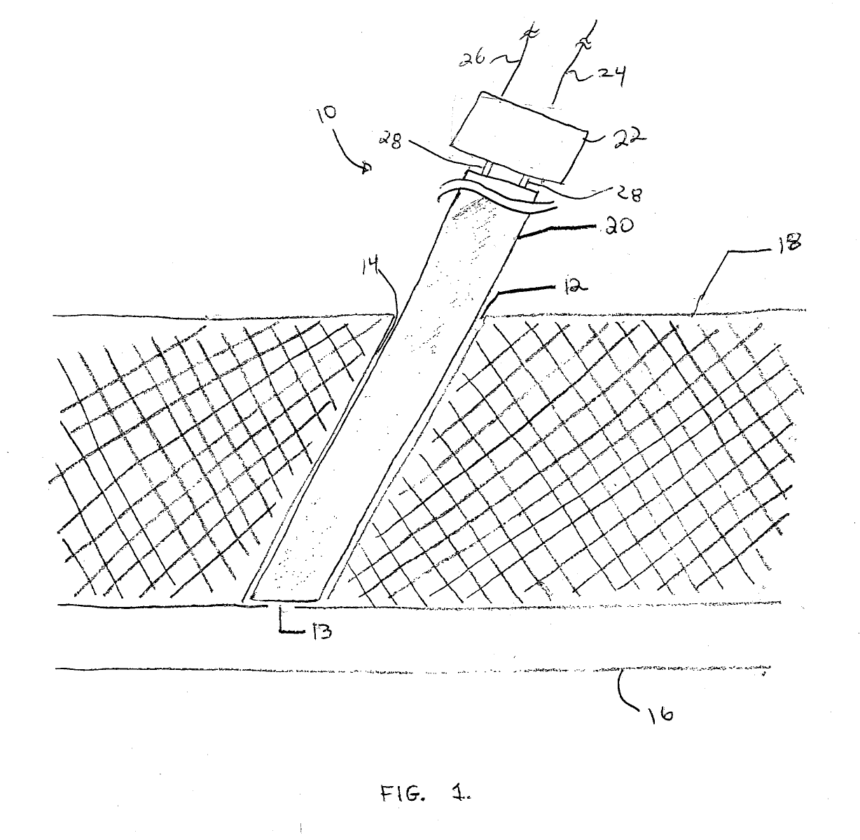

After a procedure, such as counterpulsation, has been completed, the sheath must be removed and the wound closed or sealed. FIG. 1 illustrates a side view of the sealing device 10 of the present invention specifically designed to seal such a wound. Sealing device 10 is inserted through insertion site 12 and tissue tract 14 of a patient 18, all shown in longitudinal cross section, such that it contacts or is adjacent an outer wall of blood vessel 16 but does not enter blood vessel 16. Sealing device 10 comprises an elongated cannula 20 and a actuation means 22 from which extend a proximal end of a barbed tissue connector means 24 and a snare means 26. Barbed tissue connector means 24 and a snare means 26 are at least partially disposed within cannula 20. A pair of hollow tubes 28 are disposed within cannula 20 and are connected on a proximal end to actuation means 22.

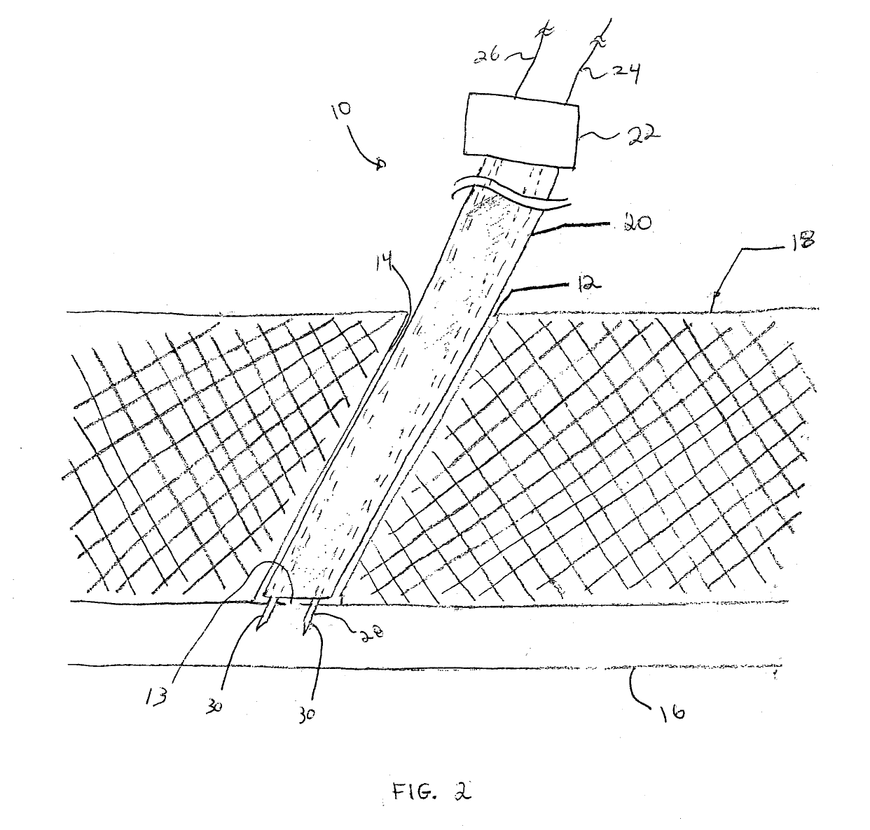

FIG. 2 illustrates the state of affairs upon completion of the next sealing step. While holding cannula 20 steady, actuation means 22 is forced towards cannula 20 which in turn shifts hollow tubes distally forcing distal portions 30 of tubes 28 through blood vessel 16 on opposing sides of puncture 13. Note that actuation means 22 may comprise any mechanical or electrical device capable of shifting tubes 28 relative to cannula 20.

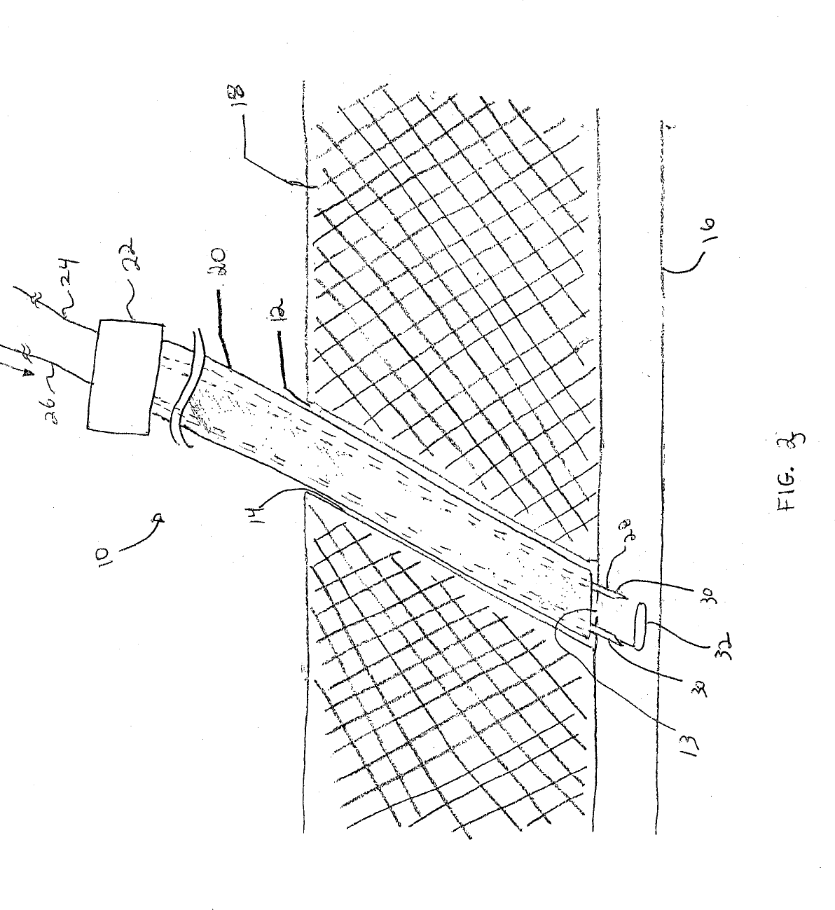

FIG. 3 illustrates the state of affairs after the next sealing step, deployment of snare means 26. Shifting snare means 26 proximally forces snare 32 at a distal end of snare means 26 out of one of the hollow tubes 28. Note that snare means may comprise a wire or another elongate structure, such as a tube.

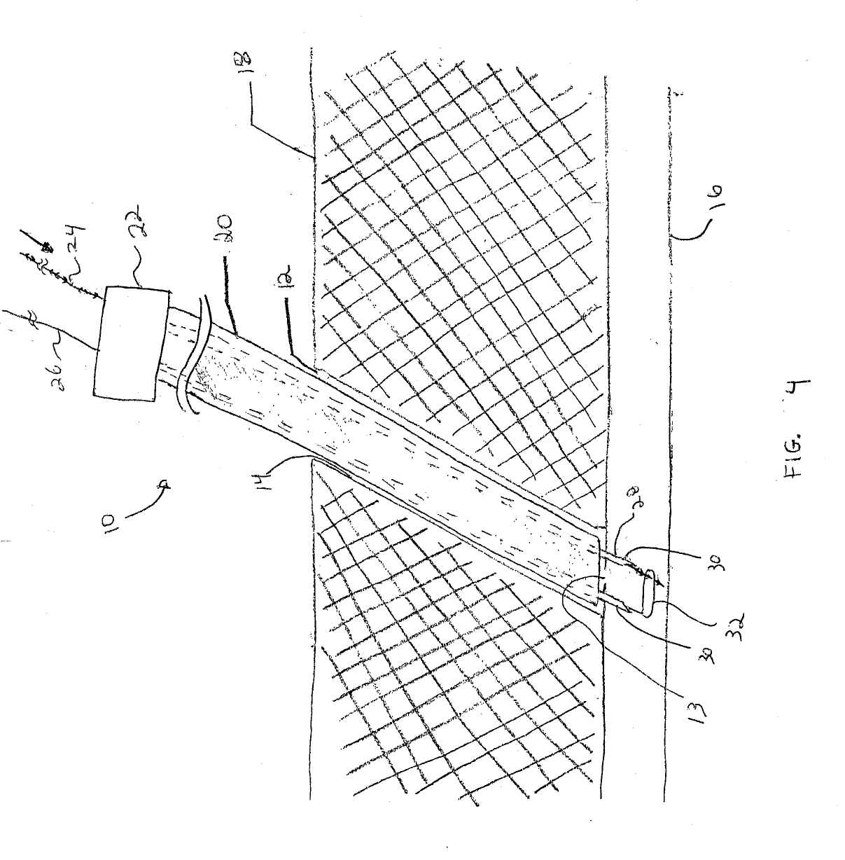

FIG. 4 illustrates the state of affairs after the next sealing step, deployment of the barbed tissue connector 24. Shifting barbed tissue connector 24 proximally forces a proximal end out of one of the hollow tubes 28 at least partially through snare 32.

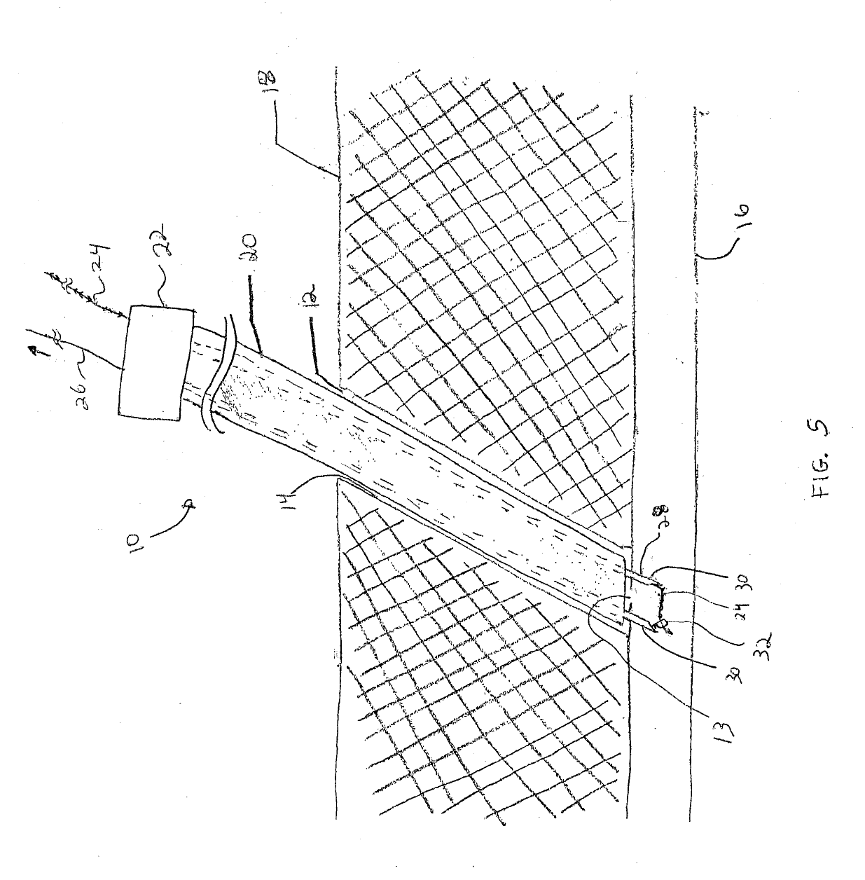

FIG. 5 illustrates the state of affairs after the next sealing step, snaring the barbed tissue connector 24. Snare means 26 is pulled proximally so as to tighten the snare 32 around barbed tissue anchor 24.

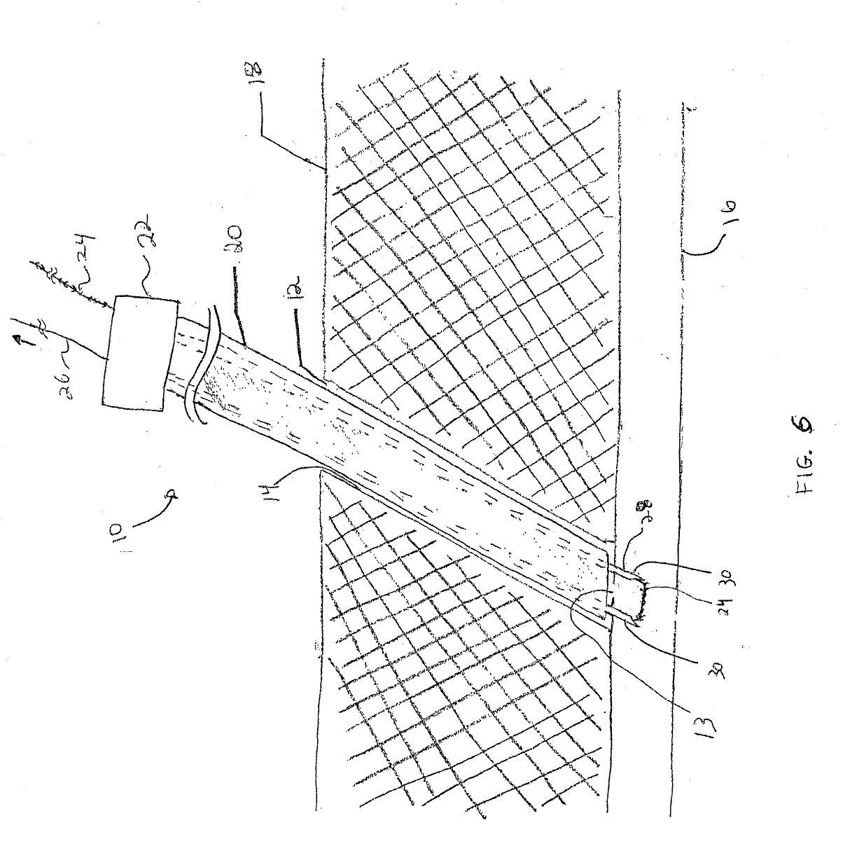

FIG. 6 illustrates the state of affairs after the next sealing step, retraction of snare means 26. Snare 32 is now disposed within the hollow tube 28 from which it originally came. Barbed tissue connector 24 now spans from one hollow tube 28 to the other.

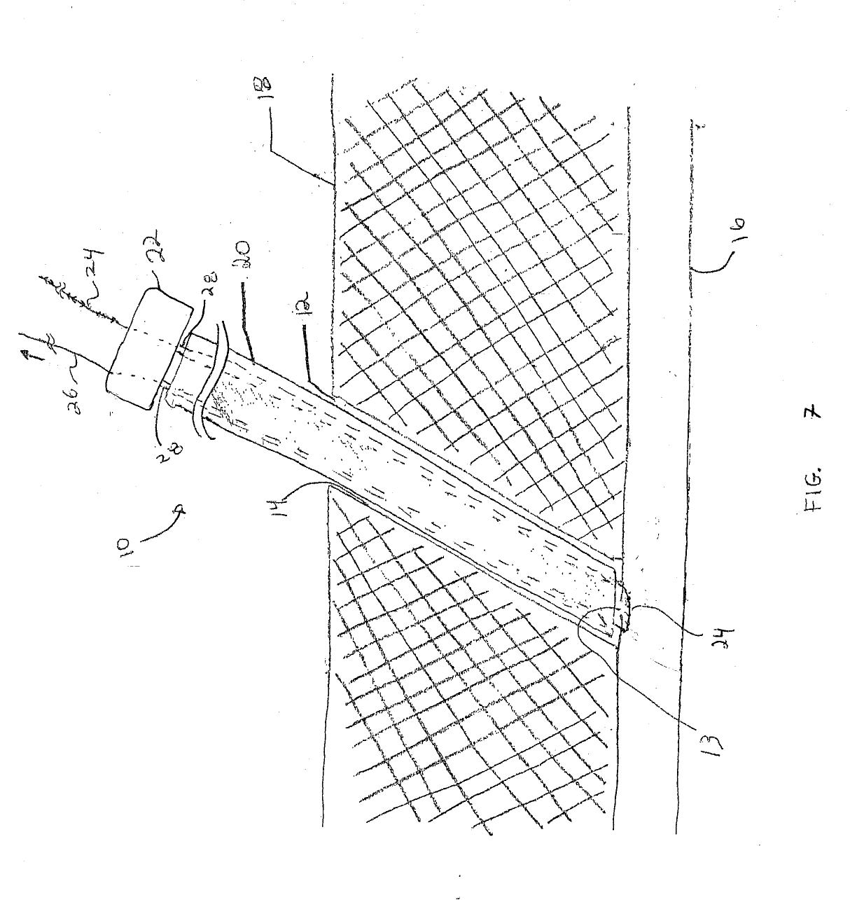

FIG. 7 illustrates the state of affairs after the next sealing step, retraction of hollow tubes 28. Actuation means 22 is activated or shifted proximally, while holding cannula 20 steady, so as to withdraw hollow tubes 28 from blood vessel 16, leaving only barbed tissue connector 24 which has been passed through opposing walls of puncture 13.

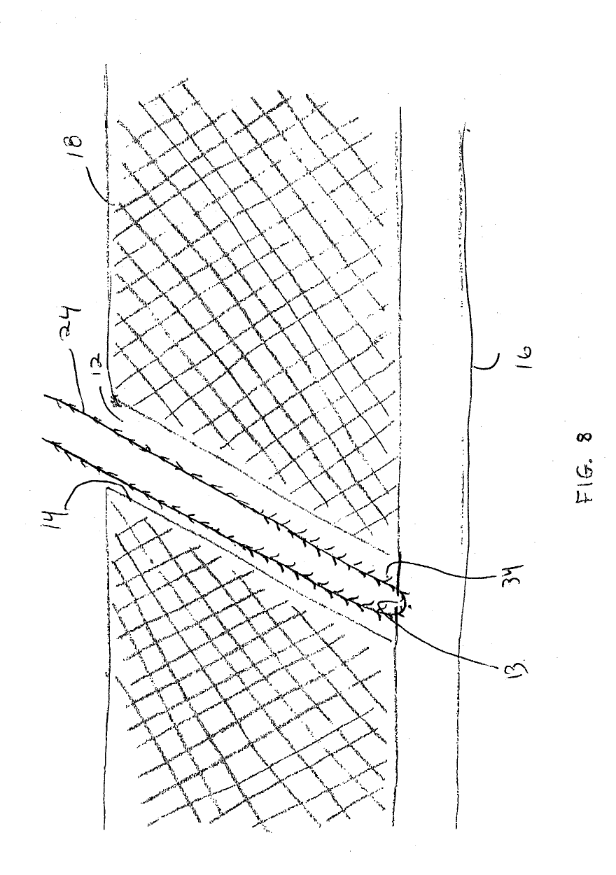

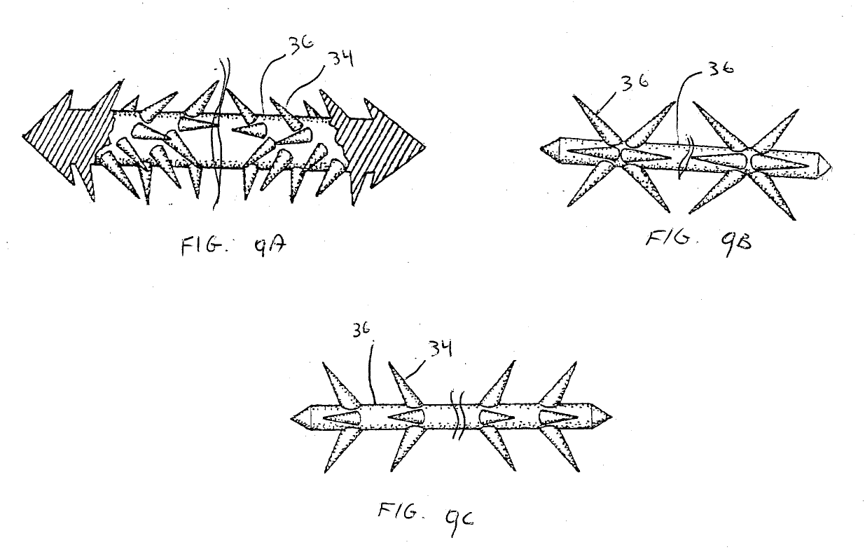

FIG. 8 illustrates the state of affairs after the next sealing step, removal of cannula 20 and closing puncture 13. By applying tension to the barbed tissue connector 24, the physician can effect the approximation of edges of puncture 13. Barbed tissue connector 24 may optionally have a anchoring means, such a toggle or specifically barbed configuration, on one end so as to prevent the tension applied by the physician from pulling barbed tissue connector 24 all the way through blood vessel 16. Unlike existing suturing devices barbed tissue anchor 24 of the present invention does not need to be knotted to maintain puncture edges in an approximated configuration. Barbs 34 (FIGS. 9A-9C) on the barbed tissue anchor 24 anchor in blood vessel 16 preventing approximated puncture 13 from opening. As a final step, the physician may trim the excess ends of barbed tissue anchor 24. Note that barbed tissue connector 24 maybe be barbed along its entire length or along selected portions, such as the portion used to effect closure of puncture 13.

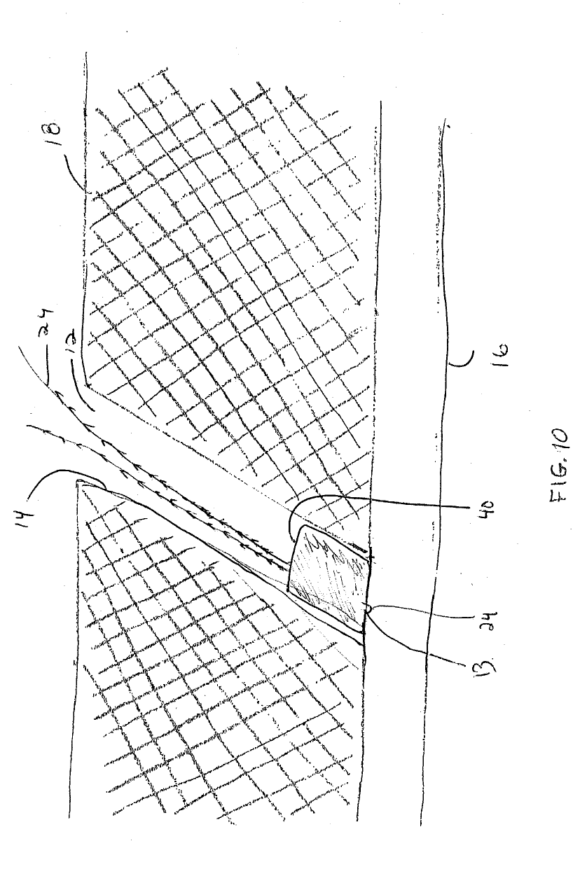

The physician may optionally apply a hemostasis means in tissue tract 14 over puncture 13 to further assure an effective seal of puncture 13. In one embodiment, a collagen plug 40 may be disposed over barbed tissue anchor 24, see FIG. 10. Barbed tissue anchor 24 prevents the hemostasis means or collagen plug 40 from entering blood vessel 16. Collagen plug 40 may be deployed using any deployment means known in the art, including manual placement; however, it is preferred to use Datascope Corp.'s VasoSeal® product, or a variation thereof, as disclosed in U.S. Pat. Nos. 5,871,501, 5,853,421, 5,830,130, 5,741,223, 5,728,122, 5,725,498, 5,591,204, 5,437,631, 5,391,183, and 5,310,407, all herein incorporated by reference in their entirety.

U.S. Pat. Nos. 5,342,376 and 6,241,747, both assigned to Quill Medical and herein incorporated by reference in their entirety, disclose various embodiments of barbed tissue anchors which are preferably employed with the present invention. FIGS. 9A-9C illustrates alternate embodiments of Quill Medical barbed tissue anchor 24. A plurality of closely spaced barbs 34 are disposed on the body of the barbed tissue anchor 24 to a predetermined location on the body. Barbs 34 may be yieldable toward body 36 to make it easier to insert the barbed tissue anchor 24 in tissue, and barbs 34 are preferably rigid in an opposite direction to hold barbed tissue connector 24 in the tissue. Note that, for the present application, barbed tissue anchors 24 do not have to have pointed ends as illustrated. Furthermore, body 36 may have alternate transverse cross sectional profiles, such as oval, square, or rectangular.

As many apparently widely different embodiments of the present invention can be made without departing from the spirit and scope thereof, it is to be understood that the invention is not limited to the specific embodiments thereof except as defined in the appended claims.

What is claimed is:

1. A method for sealing a puncture in a blood vessel comprising the step of passing a barbed tissue connector through at least two points adjacent an edge of the puncture.

2. The method as claimed in claim 1 further comprising the step of applying tension to one end of the barbed tissue connector so as to draw edges of the puncture towards each other.

3. The method as claimed in claim 2 further comprising the additional step of disposing a collagen plug over the barbed tissue anchor by passing two ends of the barbed tissue anchor through a lumen in the collagen plug.

4. A blood vessel puncture sealing device comprising a cannula, a first hollow tube, a second hollow tube, a snare means at least partially disposed within the first hollow tube, and a barbed tissue connector at least partially disposed within the second hollow tube, the first hollow tube and the second hollow tube being at least partially disposed within the cannula.

5. The blood vessel puncture sealing device further comprising an actuation means for shifting the first hollow tube and second hollow tube relative to the cannula along a longitudinal axis of the cannula.

6. The blood vessel puncture sealing means wherein the distal ends of the first hollow tube and the second hollow tube are pointed.

7. The blood vessel puncture sealing means wherein the barbed tissue connector is self-anchoring in a blood vessel wall.
